(19) United States
(12) Patent Application Publication
(10) Pub. No.: US 2025/0035456 A1
(43) Pub. Date: Jan. 30, 2025

(54) TRAVEL ROUTE PRESENTATION SYSTEM

(71) Applicant: TOYOTA JIDOSHA KABUSHIKI KAISHA, Toyota-shi (JP)

(72) Inventor: Koichiro ISOBE, Toyota-shi (JP)

(73) Assignee: TOYOTA JIDOSHA KABUSHIKI KAISHA, Toyota-shi (JP)

(21) Appl. No.: 18/434,970

(22) Filed: Feb. 7, 2024

(30) Foreign Application Priority Data
Jul. 25, 2023 (JP) 2023-120473

(51) Int. Cl. G01C 21/34; A61B 5/00

(52) U.S. Cl. CPC G01C 21/3484; A61B 5/6801; G01C 21/3476; A61B 2503/22

(57) ABSTRACT

The travel route presentation system according to the present disclosure includes: an acquisition unit that acquires predetermined information about a user; an estimation unit that estimates a health state of the user from the predetermined information about the user; a selection unit that selects at least one travel route from a current location of the vehicle to a destination based on the estimated result; and a presentation unit that presents the selected travel route. Accordingly, the travel route presentation system according to the present disclosure can propose comfortable driving by the user.

### CROSS-REFERENCE TO RELATED APPLICATION

This application claims priority to Japanese Patent Application No. 2023-120473 filed on Jul. 25, 2023, incorporated herein by reference in its entirety.

### BACKGROUND

### 1. Technical Field

The present disclosure relates to a travel route presentation system.

### 2. Description of Related Art

Japanese Unexamined Patent Application Publication No. 2019-165541 (JP 2019-165541 A) discloses a notification system that presents, based on attribute information of an occupant, a replenishment location where a vehicle is to be replenished with drive energy and that is appropriate for the occupant.

### SUMMARY

The notification system disclosed in JP 2019-165541 A merely presents the replenishment location based on information such as preferences of the occupant (user). The health condition of the occupant is not taken into consideration. Therefore, there is a possibility that the notification system disclosed in JP 2019-165541 A cannot suggest comfortable driving to the user.

The present disclosure has been made in view of the above background, and an object of the present disclosure is to provide a travel route presentation system that can suggest more comfortable driving to a user.

A travel route presentation system according to the present disclosure includes:
an acquisition unit configured to acquire predetermined information on a user;
an estimation unit configured to estimate a health condition of the user based on the predetermined information on the user;
a selection unit configured to select at least one travel route from a current location of a vehicle to a destination based on an estimation result; and
a presentation unit configured to present the selected travel route.
The user (driver) who has received the presentation can perform more comfortable driving according to the health condition of the user. That is, the travel route presentation system can suggest more comfortable driving to the user.

According to the present disclosure, it is possible to provide the travel route presentation system that can suggest more comfortable driving to the user.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram illustrating a configuration example of a travel route presentation system according to a first embodiment;

FIG. 2 is a flow chart showing an operation of the travel route presenting device provided in the travel route presentation system shown in FIG. 1;

FIG. 3 is a diagram for describing an exemplary operation of the travel route presenting device provided in the travel route presentation system shown in FIG. 1; and

FIG. 4 is a diagram for explaining an exemplary operation of the travel route presentation device provided in the travel route presentation system shown in FIG. 1.

### DETAILED DESCRIPTION OF EMBODIMENTS

Hereinafter, the present disclosure will be described through embodiments of the disclosure, but the disclosure according to the claims is not limited to the following embodiments. Further, not all of the configurations described in the embodiments are essential as means for solving the problem. For clarity of explanation, the following description and the drawings are omitted and simplified as appropriate. In the drawings, the same elements are denoted by the same reference numerals, and redundant descriptions are omitted as necessary.

### Embodiment 1

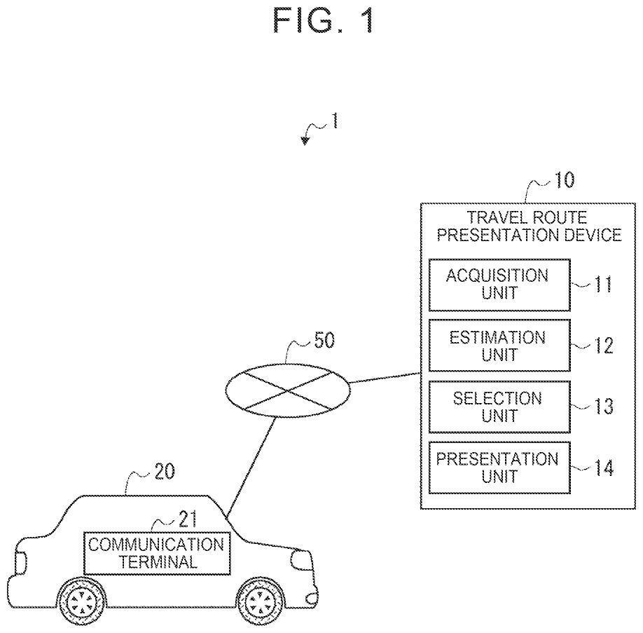

FIG. 1 is a diagram illustrating an exemplary configuration of a travel route presentation system 1 according to Embodiment 1. The travel route presentation system 1 presents a traveling route corresponding to the health condition of the user (driver). The user who has received the presentation can perform a more comfortable driving according to his/her health condition. That is, the travel route presentation system 1 can propose a comfortable driving by the user. Hereinafter, it will be described in detail.

As shown in of FIG. 1, the travel route presentation system 1 includes a travel route presentation device 10, a vehicle 20 on which the communication terminal 21 is mounted, and a network 50. The travel route presentation device 10 can also be referred to as a traveling route presentation system by itself. The travel route presentation device 10 and the communication terminal 21 are configured to be able to communicate with each other via a wired or wireless network 50.

The vehicle 20 is a vehicle such as a battery electric vehicle owned by the user. A communication terminal 21 is installed in the vehicle 20. The communication terminal 21 is, for example, a communicable car navigation device.

The travel route presentation device 10 is a device that presents a traveling route corresponding to a health state of a user. Specifically, the travel route presentation device 10 includes an acquisition unit 11, an estimation unit 12, a selection unit 13, and a presentation unit 14.

The acquisition unit 11 acquires predetermined information about the user. The predetermined information about the user is information necessary for estimating the health state of the user. For example, the acquisition unit 11 acquires at least one of a pulse rate, a heart rate, and a body temperature of the user from a wearable terminal (not illustrated) worn by the user as predetermined information about the user. Alternatively, the acquisition unit 11 acquires information input via an operation terminal (not illustrated) as predetermined information about the user. For example, the user inputs at least one of the result of the medical examination, the patient's illness, and the body temperature of the present day to the operation terminal by operating the operation terminal. The above-described wearable terminal or operation terminal may be a portable terminal such as a smartphone owned by a user. Alternatively, the acquisition unit 11 may acquire, as predetermined information about the user, the eyesight or posture of the user during driving obtained from a captured image of a camera (not shown) installed in the vehicle.

The estimation unit 12 estimates the health state of the user from the predetermined information about the user acquired by the acquisition unit 11. Here, a plurality of combinations of predetermined information about the user and the health state of the user may be stored in a database (not shown). In this case, the estimation unit 12 can extract, from the database, information related to the health state of the user corresponding to the predetermined information related to the user acquired by the acquisition unit 11, and use the extracted information as an estimation result.

The selection unit 13 selects at least one travel route from the current location of the vehicle 20 to the destination based on the health state of the user estimated by the estimation unit 12. Specifically, when the estimation unit 12 estimates that the health state of the user is lower than normal, the selection unit 13 selects a traveling route suitable for restoring the health state of the user. The predetermined area suitable for restoring the health state of the user is, for example, an area in which a predetermined hospital such as a family hospital is located, an area with good scenery, an area where the vehicle 20 can be stopped and rest, or the like.

For example, when the estimation unit 12 estimates that the physical condition of the user is not excellent or that the user's illness has occurred, the selection unit 13 selects, as an example, a travel route traveling in the vicinity of an area where a predetermined hospital such as a primary hospital is located. Alternatively, in a case where the estimation unit 12 estimates that the user is tired, the selection unit 13 may select, as an example, a travel route traveling in the vicinity of an area where a rest can be performed.

Here, a plurality of combinations of the health state of the user and information on a predetermined area suitable for restoring the health state of the user may be further stored in a database (not shown). In this case, the selection unit 13 may extract information about a predetermined area corresponding to the health state of the user from the database and then select a travel route traveling in the vicinity of the extracted predetermined area.

In addition, when the vehicle 20 is a battery electric vehicle and the destination of the vehicle 20 is a charging spot (a charging facility or a charging station), it is preferable that a combination of the user's health status and information on a predetermined area such as a hospital that is juxtaposed to the respective charging spots located within a predetermined range from the current location of the vehicle 20 is further stored in a database. In this case, the selection unit 13 may select a travel route to the set destination after extracting a charging spot in which a predetermined area corresponding to the health state of the user is provided side by side from the database and setting the charging spot as the destination.

The presentation unit 14 presents information of the travel route from the current location of the vehicle 20 to the destination, which is selected by the selection unit 13. For example, the presentation unit 14 causes the monitor of the communication terminal 21, which is a car navigation device, to display information on the travel route from the current location to the destination of the vehicle 20, which is selected by the selection unit 13. Alternatively, the presentation unit 14 may output the information of the travel route from the current location to the destination of the vehicle 20, which is selected by the selection unit 13, from the speaker mounted on the vehicle 20. Alternatively, the presentation unit 14 may display or notify information of the travel route from the current location to the destination of the vehicle 20, which is selected by the selection unit 13, to a portable terminal such as a smartphone owned by the user.

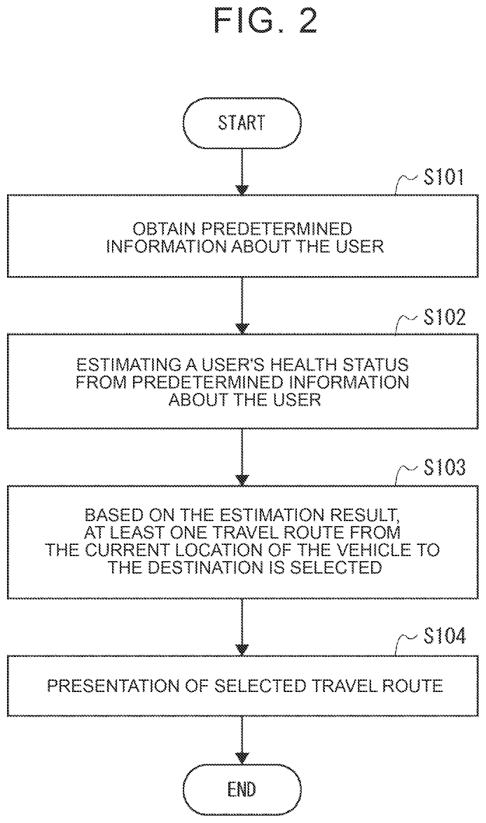

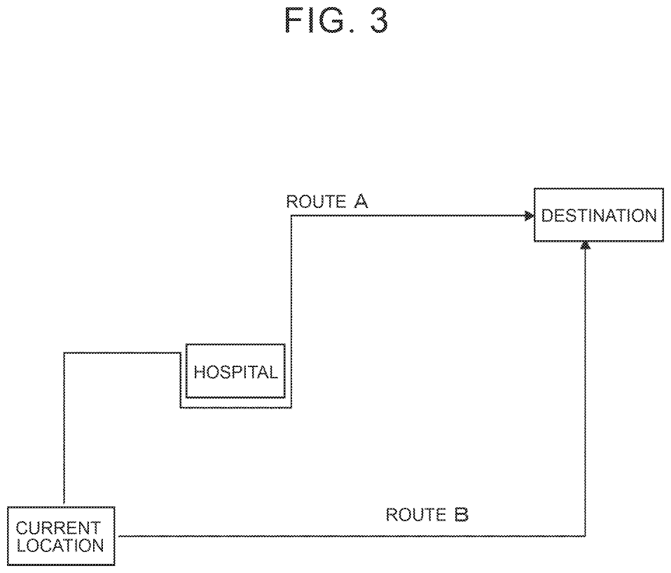

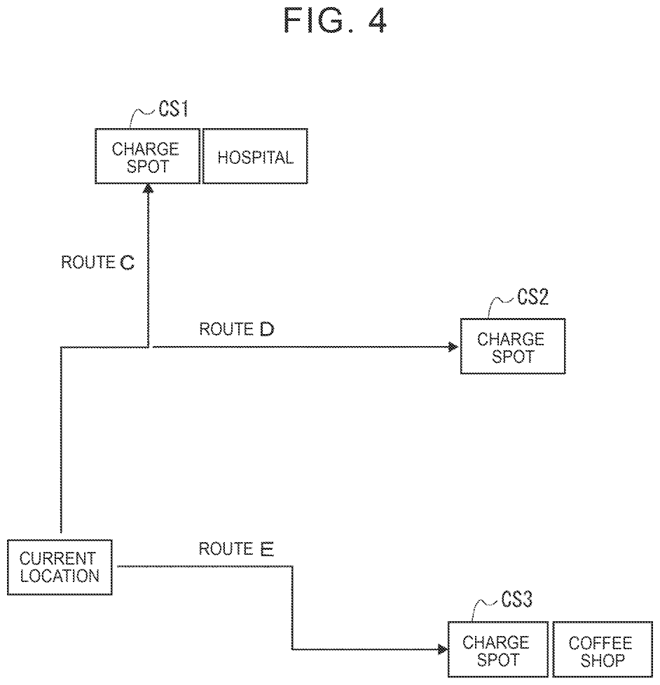

Next, the operation of the travel route presentation device 10 will be described using FIG. 2 to FIG. 4. FIG. 2 is a flow chart illustrating an operation of the travel route presentation device 10. FIG. 3 and FIG. 4 are diagrams for explaining an exemplary operation of the travel route presentation device 10.

First, the travel route presentation device 10 acquires predetermined information about the user (S101). Specifically, the travel route presentation device 10 acquires information necessary for estimating the health state of the user.

Thereafter, the travel route presentation device 10 estimates the user's health condition based on the obtained predetermined information about the user (S102).

Thereafter, the travel route presentation device 10 selects a traveling route corresponding to the estimated healthy condition of the user among one or more traveling routes from the current location of the vehicle 20 to the destination (S103). Specifically, the travel route presentation device 10 selects a traveling route that travels in the vicinity of a predetermined area suitable for recovering the estimated health state of the user from one or more traveling routes from the current location of the vehicle 20 to the destination.

In an example of FIG. 3, there are two travel routes A, B as travel routes from the present location to the destination of the vehicles 20. For example, in a case where it is estimated that the health condition of the user is not excellent, the travel route presentation device 10 selects the traveling route A traveling in the vicinity of the area where the primary hospital is located. In addition, when it is estimated that the health condition of the user is good, the travel route presentation device 10 selects the traveling route B having a short traveling distance.

In an example of FIG. 4, the vehicle 20 is a battery electric vehicle and the destination of the vehicle 20 is a charge spot. In this case, the travel route presentation device 10 selects the traveling route C after setting, as the destination, a charging spot CS1 in which a predetermined area such as a hospital or the like suitable for restoring the healthy condition of the user is juxtaposed among the three charging spot CS1 to CS3 located within a predetermined range from the present position of the vehicle 20.

Thereafter, the travel route presentation device 10 presents information regarding the selected traveling route (S104). For example, the travel route presentation device 10 displays information on the traveling route from the current location to the destination of the selected vehicle 20 on the monitor of the communication terminal 21 that is the car navigation device.

As described above, the travel route presentation system 1 according to the present embodiment presents a traveling route corresponding to the health state of the user (driver). The user who has received the presentation can perform a more comfortable driving according to his/her health condition. That is, the travel route presentation system 1 can propose a comfortable driving by the user.

In addition, some or all of the processes of the travel route presentation device 10 can be realized by causing a Central Processing Unit (CPU) to execute a computer program.

The programs described above include instructions (or software code) that, when loaded into a computer, cause the computer to perform one or more of the functions described in the embodiments. The program may be stored in a non-transitory computer-readable medium or a tangible storage medium. By way of example, and not limitation, computer-readable media or tangible storage media include Random-Access Memory (RAM), Read-Only Memory (ROM), flash memory, Solid-State Drive (SSD) or other memory techniques, CD-ROM, Digital Versatile Disc (DVD), Blu-ray disk or other optical disk storage, magnetic cassettes, magnetic tape, magnetic disk storage or other magnetic storage devices. The program may be transmitted on a transitory computer readable medium or a communication medium. By way of example, and not limitation, transitory computer-readable media or communication media include electrical, optical, acoustic, or other forms of propagated signals.

Some or all of the above-described embodiments may be described as the following supplementary notes, but are not limited thereto.

### Appendix 1

The travel route presentation system includes:
Obtain predetermined information about the user,
Estimating a health condition of the user from predetermined information about the user,
Based on the estimated result, at least one travel route from the current location of the vehicle to the destination is selected.
Present the selected travel route,
A method of presenting a travel route.

### Appendix 2

A process of obtaining predetermined information about a user,
A processing which presumes the aforementioned user's health condition from predetermined information about the aforementioned user,
A process of selecting at least one travel route from a current location of a vehicle to a destination based on the estimated result,
A process of presenting a selected travel route,
A control program that causes a computer to execute.

What is claimed is:

1. A travel route presentation system comprising:
an acquisition unit configured to acquire predetermined information on a user;
an estimation unit configured to estimate a health condition of the user based on the predetermined information on the user;
a selection unit configured to select at least one travel route from a current location of a vehicle to a destination based on an estimation result; and
a presentation unit configured to present the selected travel route.

2. The travel route presentation system according to claim 1, wherein the acquisition unit is configured to acquire at least one of a pulse rate, a heart rate, and a body temperature of the user as the predetermined information on the user from a wearable terminal worn by the user.

3. The travel route presentation system according to claim 1, wherein the acquisition unit is configured to acquire information input via an operation terminal as the predetermined information on the user.

4. The travel route presentation system according to claim 1, further comprising a database configured to store a plurality of combinations of a health condition of the user and information on a predetermined area suitable for restoring the health condition of the user, wherein the selection unit is configured to extract information on a predetermined area associated with the health condition of the user estimated by the estimation unit from the database and then select a travel route for traveling near the extracted predetermined area.

5. The travel route presentation system according to claim 1, wherein:
the vehicle is a battery electric vehicle; and
the selection unit is configured to further select, as the destination, a charging facility with a predetermined area associated with the health condition of the user estimated by the estimation unit from among a plurality of charging facilities.
